(19) United States
(12) Patent Application Publication
(10) Pub. No.: US 2019/0232019 A1
(43) Pub. Date: Aug. 1, 2019

(54) STRICTURE-DILATED URINARY CATHETER

(71) Applicant: JW ICU MEDICAL LIMITED, Weihai (CN)

(72) Inventor: Dongsheng DONG, Beijing (CN)

(21) Appl. No.: 16/339,208

(22) PCT Filed: Jan. 17, 2018
(86) PCT No.: PCT/CN2018/073022

§ 371 Date: Apr. 3, 2019

(30) Foreign Application Priority Data
May 17, 2017 (CN) 201710345800.0

(51) Int. Cl. A61M 25/00; A61M 29/02; A61M 25/10

(52) U.S. Cl. CPC A61M 25/0068; A61M 29/02; A61M 25/0026; A61M 2210/1089; A61M 25/0043; A61M 2202/0496; A61M 2025/0057; A61M 25/10

(57) ABSTRACT

In clinical practice, the procedure of urinary catheterization may cause severe irritation to urethra and bladder. In addition, physiological and pathological stricture and curvature of human urethra will increase the difficulty of urinary catheterization and aggravate the injury of lower urinary tract during insertion. The injury of lower urinary tract may induce or aggravate lower urinary tract infection and further affect the lower urinary tract or even the systemic organ function. At present, clinical solutions to urethral stricture comprise: The use of a stronger tip or metal probe or guide wire increases the external force to push forward, resulting in severe contusion of the urethra intima and susceptibility to infection; another method is to coat the outer surface of the urinary catheter with lubricating oil or lubricant coating, but using it alone is almost ineffective for urethral stricture. The utility model provides a stricture-dilated urinary catheter, which can be used for filling the dilatable part of the catheter tip thin segment in a targeted manner during the insertion, and with the uniform coating of lubricating substances, thereby improve the success rate of the insertion and minimize the risk of injury and infection.

### FIELD OF THE INVENTION

The utility model relates to a stricture-dilated urinary catheter, belonging to the technical field of medical devices.

### BACKGROUND OF THE INVENTION

Urinary catheter is the most common medical device in clinic, which can drain urine from the bladder to the outside after it is inserted into the body via the urethra, and can be removed after one urine drainage, or can be retained for multiple drainage for prolonged period.

However, the procedure of urinary catheterization itself may cause severe irritation to urethra and bladder. In addition, physiological and pathological stricture and curvature of human urethra will increase the difficulty of urinary catheterization and aggravate the injury of lower urinary tract during insertion. The injury of lower urinary tract may induce or aggravate lower urinary tract infection and further affect the lower urinary tract or even the systemic organ function.

The female urethra is much shorter than the male urethra, but because the structure around the external urethral orifice is complicated, even if the urinary catheter is not inserted, females are more susceptible to urinary tract infections. Therefore, female urethral catheterization patients are likely to have urethral stricture due to urinary tract infection before catheterization. No matter male or female, in the existing clinical procedure of urethral catheterization, after the sterilization of the external urethral orifice and its surrounding tissues, usually one hand to fix the external urethral orifice, and use the other hand to hold the sterile urinary catheter directly or with tweezers, hemostatic forceps, etc., and slowly insert it from the external urethral orifice. When the urethra is bent or accompanied by stricture, it often needs to be inserted with force, which may lead to more serious injury to the urethra. However, when the resistance is too high to pass through, it is often necessary to apply lubricating oil and use a thicker and harder urethral probe to dilate the urethra, and then insert the catheter after dilating, which will bring great pain to the patient and damage the structure of the lower urinary tract, and the lubricating oil will be trapped by most of the urethral intima in the course of the catheter's travel, and there will be little left when it reaches the stricture. Even if the catheter with super-smooth coating is used, the simple lubrication effect is still not good when urethral stricture occurs.

The existing clinical catheterization has the following problems in the insertion procedure:

1. When the urinary catheter tip end travels to the stricture of the urethra, it usually needs to increase the force to push it forward and shear with the urethra at the stricture. Even if it passes through the stricture, the urethral intima will suffer severe contusion and create conditions for microbial infection.

2. The application of lubricating oil or lubricant coating to the outer surface of the urinary catheter alone is almost ineffective for urethral stricture.

### SUMMARY

In order to solve the above problems, the utility model provides a stricture-dilated urinary catheter.

The object of the utility model is achieved by:

1. A stricture-dilated urinary catheter, which is a hollow tube made of solid material having an inner surface and an outer surface, comprising a urinary catheter tip that enters the bladder, a urinary catheter tail that is left outside the body and a urinary catheter middle section connecting the tip and the tail for use, in which, the tube hollow is a urinary catheter inner cavity, the urinary catheter tip is provided with a urine inlet and the urinary catheter tail is provided with a urine outlet; a nipple-shaped flexible and hollow thin segment is connected to the outer surface of the urinary catheter tip end, the hollow inside the thin segment is a thin segment inner cavity and the thin segment inner cavity may have no air or volume in the non-dilated state, one side of the thin segment connected to the outer surface of the urinary catheter tip end is a thin segment bottom, and the other side is a thin segment top; the thin segment bottom is connected with the outer surface of the tip end and is the connecting part of the thin segment, and the rest part including the thin segment top is a thin segment dilatable part; the thin segment inner cavity is specifically formed by the inner surface of the thin segment dilatable part and a portion of the outer surface of the urinary catheter tip end covered by the thin segment dilatable part; the urinary catheter thin segment top is closed to the outside by the blind end, and the average outer diameter of the part between the thin segment top farthest point and the urinary catheter tip end farthest point is smaller than the maximum outer diameter of the urinary catheter tip end; the thin segment inner cavity is connected to the outside through at least one route on the catheter wall, i.e., the thin segment inner cavity route. The thin segment inner cavity route has at least one opening connecting with the thin segment inner cavity, which is the thin segment inner cavity route inner opening and at least one thin segment inner cavity route outer opening at the urinary catheter middle section or tail that can be connected with the external space.

The thin segment is structurally an extension of the urinary catheter tip end. The direction of the said extension can be either along the catheter tip centerline, or parallel to the centerline, or at a certain angle to the centerline, i.e. biased extension, sometimes the thin segment of biased extension is more easily to get into the urethra at the corner. When the catheter travels in the urethra and encounters the stricture, the tip end with a larger outer diameter cannot pass through, while the thin segment with a smaller outer diameter can enter the stricture of the urethra. At this time, use a syringe, pump, elastic balloon or other tools to fill air and/or fluid of certain pressure from outside into the thin segment inner cavity via the thin segment inner cavity route, so that the thin segment is dilated and maintained for a certain period of time, and the generated thrust is vertically applied on the urethral intima at the stricture to dilate the urethra, and then the air and/or liquid into the thin segment inner cavity are/is sucked out from the outside. After the dilatation of the thin segment is relieved, continue to push the catheter forward and the catheter tip end is easy to pass through the dilated urethral portion.

In order to facilitate the filling and dilatation of the thin segment for the purpose of dilating urethral stricture, in addition to the main drainage branch connected with the urinary catheter inner cavity, the urinary catheter tail further comprises a collateral branch, i.e. thin segment filling collateral branch, connected with the thin segment inner cavity route. A hollow elastic balloon is connected to the end of the collateral branch, and the elastic balloon inner cavity is connected with the thin segment inner cavity route outer opening.

In order to further facilitate the procedure, a hollow elastic balloon is sleeved on the middle section near the urinary catheter tail or the outer surface of the main drainage branch, and the elastic balloon inner cavity is connected with the thin segment inner cavity route outer opening, that is, the thin segment inner cavity route outer opening is provided on the urinary catheter outer surface closed and covered by the elastic balloon.

The elastic balloon used for filling the thin segment inner cavity is provided with at least one elastic balloon external opening which can be covered by fingers, and the air and/or liquid inside the elastic balloon inner cavity are/is connected with the outside through this opening. The elastic balloon inner cavity for filling the thin segment inner cavity may be filled with liquid and/or air in advance.

When it is used, press the elastic balloon while covering the elastic balloon external opening with fingers. Since the elastic balloon external opening is closed by fingers, the air and/or liquid in the elastic balloon inner cavity is pressed to the thin segment inner cavity through the thin segment inner cavity route and inflated to enlarge the strictured urethra. The elastic balloon may be restored if the pressing is stopped, and the air and/or liquid entering the thin segment inner cavity will be sucked back into the elastic balloon inner cavity, or the air and/or liquid entering the thin segment inner cavity will be pushed back into the elastic balloon inner cavity due to the elastic restoring force of the thin segment, or the finger's closure of the elastic balloon external opening will be released, so that the air and/or liquid in the elastic balloon inner cavity will be connected with the outside, and finally the thin segment will recover from the dilated state. However, due to the presence of the elastic balloon external opening during indwelling of the urinary catheter, when the elastic balloon is pressed due to involuntary movement of the lower limbs or local medical care operation, the air and/or liquid in the elastic balloon inner cavity can flow out of the elastic balloon external opening, thus avoiding possible stimulation of the bladder intima by the dilatation of the thin segment located in the bladder at this time.

Further, another external opening is provided on the opposite side of one elastic balloon external opening. The two openings can be simultaneously covered by two fingers moving relative to each other when used. When the elastic balloon is pressed due to involuntary movement of lower limbs or local medical care operation during indwelling of the urinary catheter, the reliability of air and/or liquid in the elastic balloon inner cavity that can flow out from the two elastic balloon external openings is greatly improved compared with the one due to the presence of the two opposite elastic balloon openings.

Further, in order to ensure that the elastic balloon outside the body does not fill the thin segment inside the body due to accidental compression during indwelling of the urinary catheter, a switching part which is not easy to deform under pressure and can move relative to the outer wall of the urinary catheter is provided on or near the elastic balloon, and a protrusion which can close another external opening of the elastic balloon is provided on the switching part; when the position of the switching part is moved, the closing protrusion thereon can release the closing of another external opening of the elastic balloon, and enable the elastic balloon inner cavity to connect with the outside through a hidden route on the switching part. The said hidden route of the switching part refers to an external connection route that presses the outer surface of the elastic balloon and/or the outer surface of the switching part not to be closed, that is, a route that is not in the easily deformable part and/or the easily accessible part.

A hidden route can be selected at the end face of the switching part that can be rotated or pushed and pulled. The outer surface of the elastic balloon and/or the outer surface of the switching part may be provided with marks indicating the state of the switching part, which may be printed marks or concave-convex marks of local materials.

In order to ensure that the thin segment is easily filled and dilated, at least one inward urinary catheter tip end annular recess is provided on the outer surface of the portion where the urinary catheter tip end is covered by the thin segment dilatable part, and a thin segment inner cavity route inner opening is provided in the urinary catheter tip end annular recess.

Further, at least two inward urinary catheter tip end annular recesses are provided on the outer surface of the portion where the urinary catheter tip end is covered by the thin segment dilatable part, and at least one connecting slot recessed in the outer surface of the tip end is provided between these annular recesses, and a thin segment inner cavity route inner opening is provided in these annular recesses and/or connecting slot.

In order to increase the external strength of the thin segment during its travel, a part of the catheter tip end extends into the thin segment inner cavity. The extension of the catheter tip end increases the strength of the thin segment, making it easier for the thin segment to enter the narrow section of the urethra.

In order to accurately and evenly apply lubricant and/or drugs, at least one inward thin segment annular recess is provided on the outer surface of the thin segment dilatable part, and the inner space of the thin segment annular recess can store lubricant and/or drugs, and the stored lubricant and/or drugs can be annularly coated on the urethral intima when the thin segment is filled and dilated.

In order to avoid the loss of lubricant and/or drugs before reaching the application site, at least one inward urinary catheter tip end annular conformal recess is provided on a portion where the urinary catheter tip end is covered by the thin segment dilatable part, and the thin segment dilatable part is also provided with a thin segment annular recess at this annular conformal recess. More lubricant and/or drugs can be stored in the inner space of this annular recess of the thin segment.

### The Beneficial Effects of the Utility Model are:

1. When the catheter tip end travels to the stricture of the urethra, the thin segment can be probed into the stricture area in advance, and pressing the elastic balloon can dilate the thin segment and push away the urethral intima at the stricture in the perpendicular direction, which is helpful for the catheter to smoothly pass through the stricture and avoids the shear damage to the urethral intima caused by pushing along the horizontal direction to the greatest extent.

2. Lubricating substances and/or drugs in the annular recess of the thin segment are synchronously and accurately coated on the urethral intima at the stricture when it is dilated, and the precise lubrication at the stricture is more conducive to the smooth passage of the urinary catheter, and the directional application of drugs also provides a reliable guarantee for the treatment of urethral diseases.

### DESCRIPTION OF DRAWINGS

FIG. 1 is a schematic view of the existing urinary catheter tip entering the urethral stricture;

FIG. 2A is a structural schematic view according to Embodiment 1 of the utility model;

FIG. 2B is an enlarged structural schematic view of the thin segment according to Embodiment 1 of the utility model;

FIG. 2C is a schematic view of the deflection of the urinary catheter tip end according to Embodiment 2 of the utility model;

FIG. 3 is a schematic view of the thin segment entering the urethral stricture according to Embodiment 1 of the utility model;

FIG. 4 is a schematic view of the dilatable state of the thin segment entering the urethral stricture according to Embodiment 1 of the utility model;

FIG. 5 is a schematic structural view of the elastic balloon which can make the thin segment fill and dilate according to Embodiment 3 of the utility model;

FIG. 6 is another schematic structural view of the elastic balloon which can make the thin segment fill and dilate according to Embodiment 4 of the utility model;

FIG. 7 is a schematic view of the external opening on the elastic balloon according to Embodiment 5 of the utility model;

FIG. 8A is a schematic view of the switching part on the elastic balloon according to Embodiment 6 of the utility model;

FIG. 8B is a structural schematic view of the closed state of the switching part on the elastic balloon according to Embodiment 6 of the utility model;

FIG. 8C is a structural schematic view of the open state of the switching part on the elastic balloon according to Embodiment 6 of the utility model;

FIG. 9 is a structural schematic view of the annular recess of the urinary catheter tip end according to Embodiment 7 of the utility model;

FIG. 10A is a structural schematic view of the isolated thin segment inner cavity route inner opening in the urinary catheter tip end according to Embodiment 7 of the utility model;

FIG. 10B is a cross-sectional schematic view of the isolated thin segment inner cavity route inner opening in the urinary catheter tip end according to Embodiment 7 of the utility model;

FIG. 10C is a cross-sectional schematic view of the isolated thin segment inner cavity route inner opening under pressure in the urinary catheter tip end according to Embodiment 7 of the utility model;

FIG. 11 is a structural schematic view of one part of the urinary catheter tip end extending into the thin segment inner cavity according to Embodiment 8 of the utility model;

FIG. 12A is a structural schematic view of the inward annular recess at the outer surface of the thin segment dilatable part according to Embodiment 9 of the utility model;

FIG. 12B is another structural schematic view of the inward annular recess at the outer surface of the thin segment dilatable part according to Embodiment 9 of the utility model;

FIG. 13 is a schematic view of a synchronous conformal recess between the urinary catheter tip end and the thin segment according to Embodiment 10 of the utility model;

Reference numbers in the figures: 0. Bladder; 00. Bladder cavity; 01. Urethral wall; 010. Urethral inner cavity; 010a. Urethral inner cavity at the stricture before dilatation; 010b. Urethral inner cavity after dilatation; 1. Urinary catheter; 10. Urinary catheter inner cavity; 11. Urinary catheter inner surface; 12. Urinary catheter outer surface; 13. Urinary catheter tip; 130. Urine inlet; 131. Urinary catheter tip end; 131p. Urinary catheter tip end farthest point; 1310. Urinary catheter tip end annular recess; 1311. Thin segment inner cavity route inner opening; 1312. Thin segment inner cavity route; 1313. Thin segment inner cavity route outer opening; 1314. Urinary catheter tip end annular conformal recess; 1315. Part of the urinary catheter tip end whose outer surface is covered by the thin segment dilatable part; 1316. Part of the urinary catheter tip end extending into the thin segment inner cavity; 132. Bladder fixing balloon; 1320. Bladder fixing balloon inner cavity; 1321. Bladder fixing balloon route; 1322. Bladder fixing balloon route collateral branch external opening; 14. Urinary catheter tail; 140. Urine outlet; 141. Main drainage branch; 142. Fixing balloon filling collateral branch; 143. Thin segment filling collateral branch; 15. Urinary catheter middle section; 16. Thin segment; 160. Thin segment inner cavity; 161. Thin segment bottom; 1612. Thin segment dilatable part; 162. Thin segment top; 162p. Thin segment top farthest point; 16. Lubricating substance and/or drug; 164. Thin segment annular recess; 17. Hollow elastic balloon; 170. Elastic balloon inner cavity; 171, 172. Elastic balloon external opening; 173. Another external opening of elastic balloon; 2. Non-return valve; 3. Switching part; 30. Hidden route; 31. Closed protrusion; 32. Mark indicating the state of the switching part; 33. Indicating arrow

### DETAILED DESCRIPTION OF EMBODIMENTS

### Embodiment 1

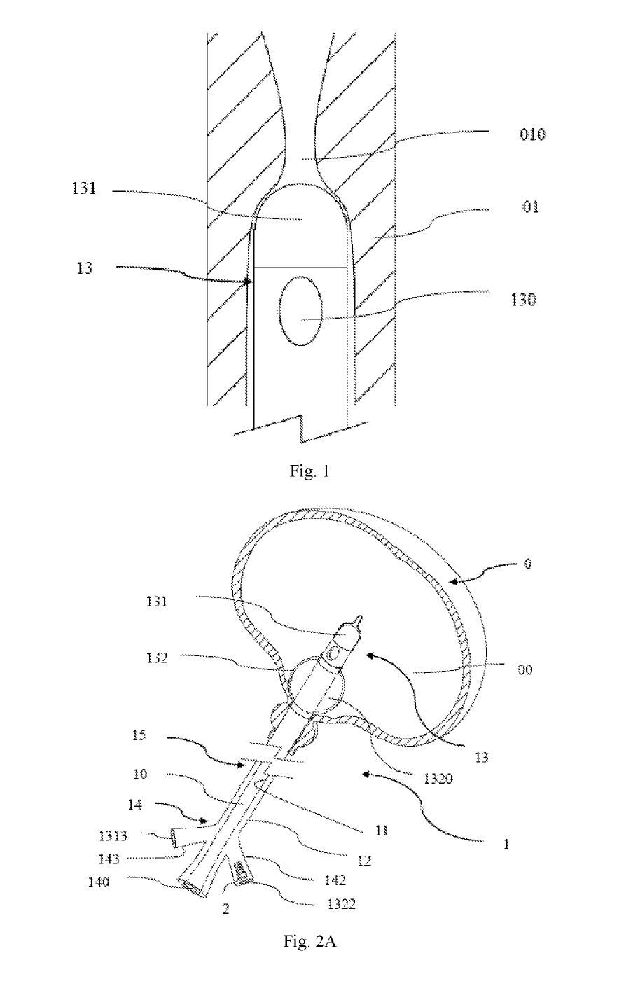

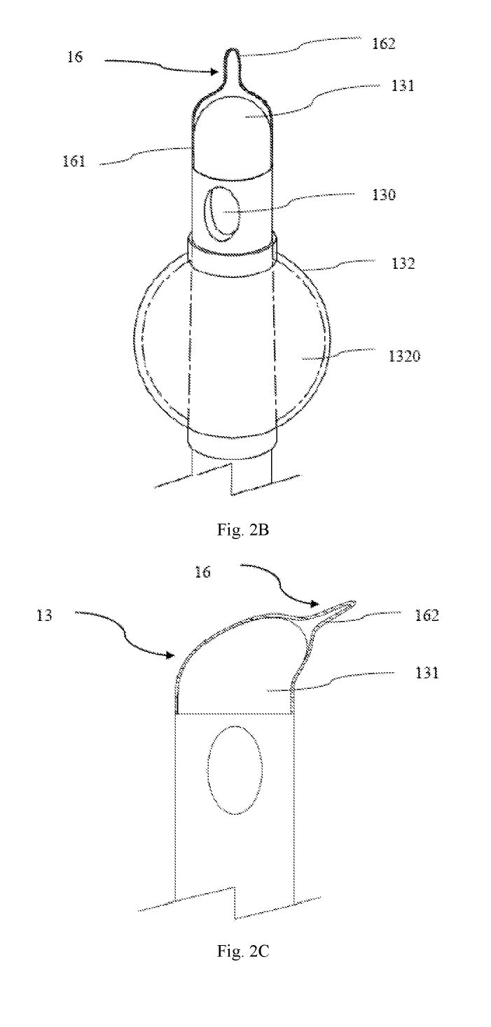

As shown in FIGS. 2A, 2B, 3 and 4, a stricture-dilated urinary catheter is a hollow tube made of solid material having an inner surface 11 and an outer surface 12, comprising a urinary catheter tip 13 that enters the bladder 0, a urinary catheter tail 14 that is left outside the body and a urinary catheter middle section 15 connecting the urinary catheter tip 13 and the urinary catheter tail 14 for use, in which, the tube inside is provided with a urinary catheter inner cavity 10 and the urinary catheter tip 13 is provided with a urine inlet 130 and the urinary catheter tail 14 is provided with a urine outlet 140; the bladder fixing balloon 132 in the bladder cavity 00 and, the fixing balloon filling collateral branch 142 of the bladder fixing balloon 132 have a bladder fixing balloon route collateral branch external opening 1322 that can be closed by a non-return valve 2 or an elastomer that can be resealed after puncture. Unlike the existing clinical catheters, a nipple-shaped flexible and hollow thin segment 16 is connected to the outer surface of the urinary catheter tip end 131, and one side of the thin segment 16 connected to the outer surface of the urinary catheter tip end 131 is a thin segment bottom 161, and the other side far away from the urinary catheter tip end 131 is a thin segment top 162; the thin segment top 162 is closed to the outside by the blind end, and the average outer diameter of the part between the thin segment top farthest point 162p and the urinary catheter tip end farthest point 131p is smaller than the maximum outer diameter of the urinary catheter tip end 131; one part of the thin segment bottom 161 is connected with the outer surface of the urinary catheter tip end 131 and is the thin segment connecting part, and the rest part including the thin segment top 162 is the thin segment dilatable part 1612; the thin segment 16 inside is provided with a thin segment inner cavity 160 which may have no air or volume but with the filling of fluid, the thin segment inner cavity 160 can be enlarged, the thin segment inner cavity 160 is specifically formed by the inner surface of the thin segment dilatable part 1612 and a part 1315 of the outer surface of the tip end 131 covered by the thin segment dilatable part 1612; the thin segment inner cavity 160 is connected to the outside through at least one route on the catheter wall, i.e., the thin segment inner cavity route 1312. The thin segment inner cavity route 1312 has at least one opening connecting with the thin segment inner cavity 160, which is the thin segment inner cavity route inner opening 1311 and at least one thin segment inner cavity route outer opening 1313 at the urinary catheter middle section 15 or the urinary catheter tail 14 that can be connected with the external space. The thin segment inner cavity route outer opening 1313 in this embodiment is located on the thin segment filling collateral branch 143 at the urinary catheter tail 14.

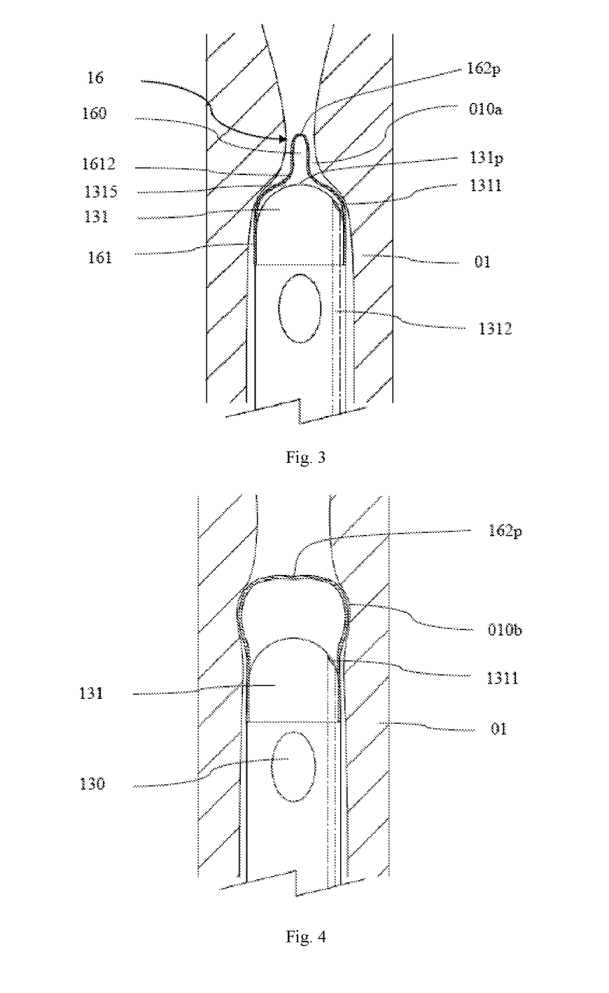

As shown in FIG. 1, when the existing urinary catheter tip enters the urethral stricture, it cannot pass through smoothly, and pushing forward may cause shear damage to the urethral intima at the stricture. For the utility model as shown in FIG. 3, when the catheter tip travels in the urethra and encounters a stricture, the urinary catheter tip end 131 having a larger outer diameter cannot pass through, while the thin segment 16 having a smaller outer diameter, especially the thin segment top 162, can enter the urethral inner cavity 010a of the stricture before dilation without causing damage to the urethra or the extent of damage is the lowest. At this time, stop advancing the urinary catheter, and as shown in FIG. 4, use a syringe, pump, elastic balloon or other tools to fill air and/or fluid of certain pressure from outside into the thin segment inner cavity 160 via the thin segment inner cavity route 1312, so that the thin segment 16 is dilated and maintained for a certain period of time, and the generated thrust from the filling and dilation of the thin segment 16 is vertically applied on the urethral intima at the stricture to dilate the urethra, and then the air and/or liquid into the thin segment inner cavity 160 are/is sucked out from the outside. After the dilatation of the thin segment 16 is relieved, continue to push the catheter forward and the urinary catheter tip end 131 is easy to pass through the dilated urethral inner cavity 010b, and on the premise of not damaging the urethra, push the catheter smoothly toward the direction of the bladder 0; continue to move forward and repeat the filling process of the above-mentioned thin segment 16 in case of recurrence of stricture until the catheter 1 is successfully inserted.

### Embodiment 2

As shown in FIG. 2C, the urinary catheter tip end 131 is biased to one side; the thin segment 16 is a functional extension of the urinary catheter tip end 131 in structure. The direction of the said extension may be either along the centerline of the urinary catheter tip 13, parallel to the centerline, or at a certain angle to the centerline, i.e. biased extension, sometimes the thin segment 16 of biased extension is more easily into the urethra at the corner. The difference between this embodiment and Embodiment 1 is that the urinary catheter tip end 131 is biased to one side and the thin segment 16 connected to it is also biased synchronously.

### Embodiment 3

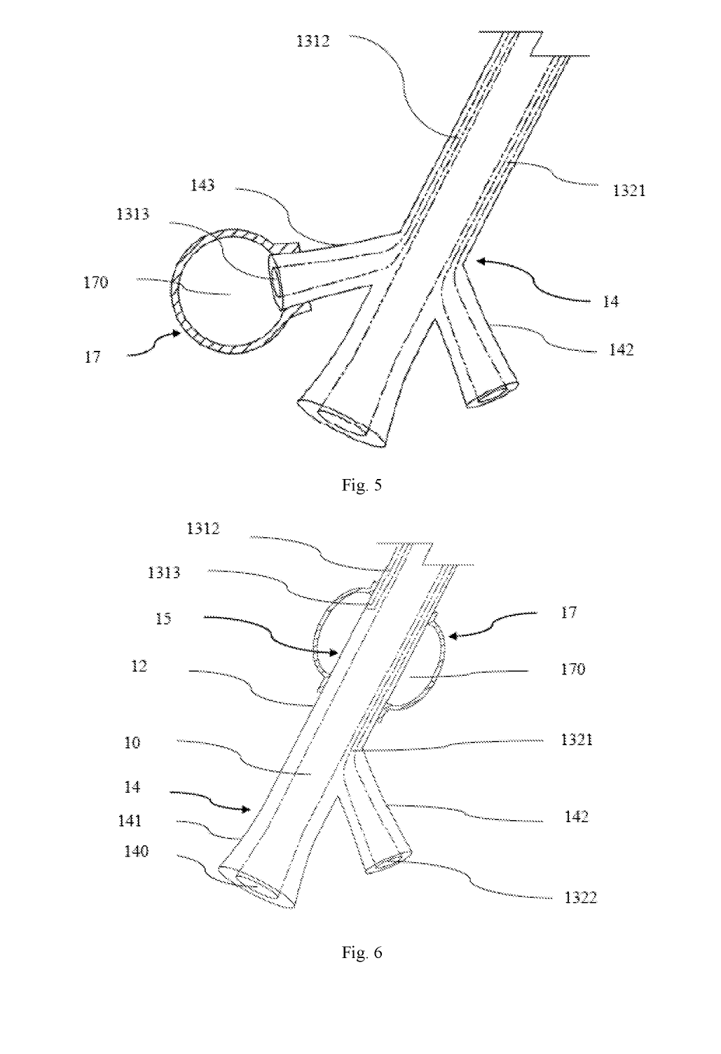

As shown in FIG. 5, the power of the utility model for filling and expanding the thin segment comes from an elastic balloon 17, which is located on the thin segment filling collateral branch 143 of the urinary catheter tail 14. The thin segment inner cavity route outer opening 1313 is provided in the elastic balloon inner cavity 170, and the elastic balloon inner cavity 170 is connected only with the thin segment inner cavity route 1312. The air in the elastic balloon 17 can be filled into the thin segment inner cavity 160 due to the external compression of the elastic balloon 17, and the thin segment 16 is dilated, and the air filled into the thin segment inner cavity 160 after pressure relief flows back into the elastic balloon inner cavity 170 due to the elastic restoring force of the elastic balloon wall 17, and the thin segment 16 is restored to its original state. The elastic balloon inner cavity 170 may also be filled with liquid substances such as sterile water and glycerol in advance.

When the urinary catheter is provided with a bladder fixing balloon 132 that can be filled and dilated and prevent the urinary catheter from prolapsing from the bladder, as shown in FIGS. 2A, 2B, 5 and 6, the bladder fixing balloon inner cavity 1320 is connected to the fixed bladder filling collateral branch 142 of the urinary catheter tail 14 through the bladder fixing balloon route 1321 on the urinary catheter wall, the bladder fixing balloon route collateral branch external opening 1322 can be sealed with an elastomer or non-return valve 2 that can be re-sealed after being punctured, and the thin segment filling collateral branch 143 and the fixed bladder filling collateral branch 142 are combined into one, i.e., the two routes pass through a common collateral branch together at the urinary catheter tail 14, and the elastic balloon 17 is located on this collateral branch, with the drawings omitted.

### Embodiment 4

As shown in FIG. 6, unlike Embodiment 3, the elastic balloon 17 that can fill and dilate the thin segment is located near the urinary catheter middle section 15 of the urinary catheter tail 14 and can be sleeved on the urinary catheter outer surface 12 or on the outer surface 12 of the main drainage branch 141. The two natural openings of the elastic balloon 17 are hermetically connected by the urinary catheter outer surface 12, and the elastic balloon inner cavity 170 is connected with the thin segment inner cavity route outer opening 1313, i.e., the thin segment inner cavity route outer opening 1313 is provided on the urinary catheter outer surface 12 that is closed and covered by the elastic balloon 17. This design offers more convenience to operate because the elastic balloon 17 to be pressed for use is located on the urinary catheter middle section 15 or the main drainage branch 141.

### Embodiment 5

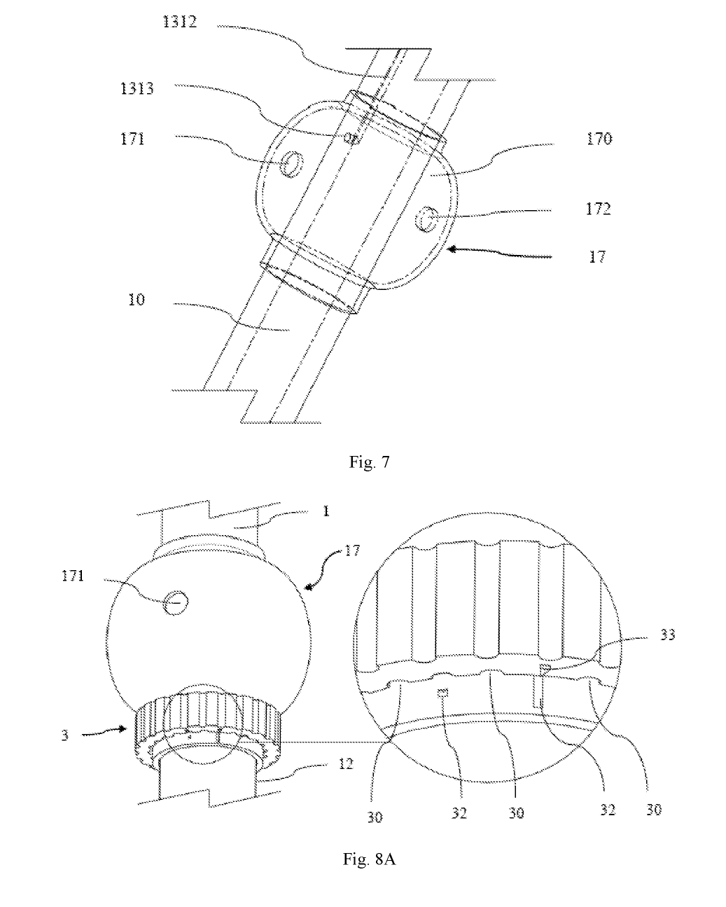

As shown in FIG. 7, the difference between this embodiment and Embodiments 3 and 4 is that the elastic balloon 17 used for filling the thin segment inner cavity 160 is provided with at least one elastic balloon external opening 171 which can be covered by fingers. The air in the elastic balloon inner cavity 170 is connected with the outside through this opening. When it is used, press the elastic balloon 17 while covering the elastic balloon external opening 171 with fingers. Since the elastic balloon external opening 171 is closed by fingers, the air in the elastic balloon inner cavity 170 is pressed to the thin segment inner cavity 160 through the thin segment inner cavity route 1312 and inflated to enlarge the strictured urethra. The elastic balloon 17 may be restored if the pressing is stopped, and the air entering the thin segment inner cavity 160 will be sucked back into the elastic balloon inner cavity 170, or the air entering the thin segment inner cavity 160 will be pushed back into the elastic balloon inner cavity 170 due to the elastic restoring force of the thin segment 16, or the finger's closure of the elastic balloon external opening 171 will be released, so that the air in the elastic balloon inner cavity 170 will be connected with the outside, and finally the thin segment 16 will recover from the dilated state. However, due to the presence of the elastic balloon external opening 171 during indwelling of the urinary catheter, when the elastic balloon 17 is pressed due to involuntary movement of the lower limbs or local medical care operation, the air and/or liquid in the elastic balloon inner cavity 170 can flow out of the elastic balloon external opening 171, thus avoiding possible stimulation of the bladder intima by the dilatation of the thin segment located in the bladder at this time.

Alternatively, an external opening 172 may be further provided on the other side, i.e. the opposite side, of the elastic balloon external opening 171, and the two openings may be simultaneously covered by two fingers moving relative to each other when in use. When the elastic balloon 17 is pressed due to involuntary movement of lower limbs or local medical care operation during indwelling of the urinary catheter, the reliability of air and/or liquid in the elastic balloon inner cavity 170 that can flow out from the two elastic balloon external openings 171, 172 is greatly improved compared with the one due to the presence of the two opposite elastic balloon openings 171, 172.

### Embodiment 6

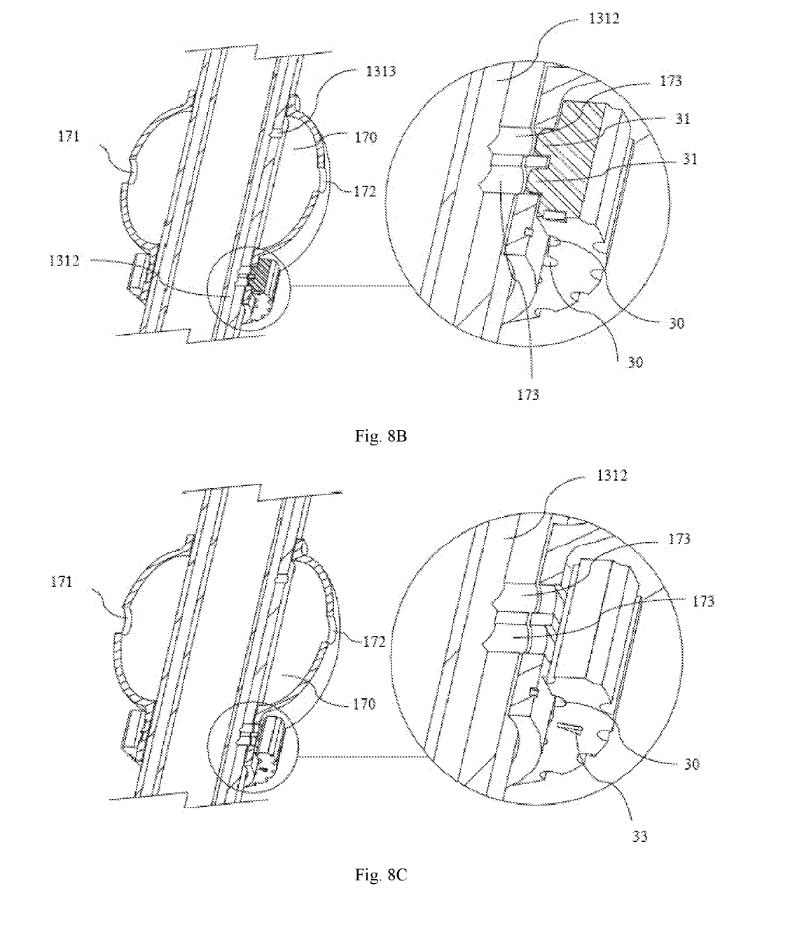

As shown in FIGS. 8A, 8B and 8C, a switching part 3 which is not easy to deform under pressure and can move relative to the catheter outer wall 12 is provided on or near the elastic balloon 17 of the utility model, and a protrusion 31 which can close another external opening of elastic balloon 173 is provided on the switching part as shown in FIG. 8B; when the position of the switching part 3 is moved, the closing protrusion 31 thereon can release the closing of another external opening of elastic balloon 173, as shown in FIG. 8C, and enable the elastic balloon inner cavity 170 to connect with the outside through a hidden route 30 on the switching part 3. The said hidden route 30 of the switching part refers to an external connection route that presses the outer surface of the elastic balloon 17 and/or the outer surface of the switching part not to be closed, that is, a route that is not in the easily deformable part and/or the easily accessible part.

In this embodiment, a hidden route 30 located at the end face of the rotatable switching part is described, and a hidden route located at the end face of the push-pull switching member may also be applied. The outer surface of the elastic balloon 17 and/or the outer surface of the switching part 3 may be provided with marks 32 indicating the state of the switching part and indicating arrows 33, which may be printed marks or concave-convex marks of local materials.

### Embodiment 7

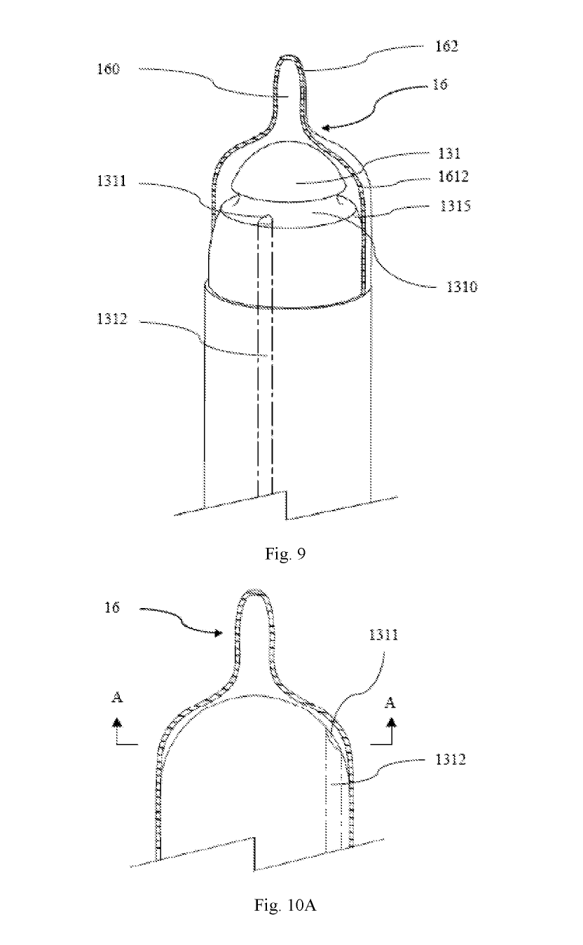

As shown in FIG. 9, at least one inward urinary catheter tip end annular recess 1310 is provided on the outer surface of the portion 1315 where the urinary catheter tip end 131 is covered by the thin segment dilatable part, and a thin segment inner cavity route inner opening 1311 is provided in urinary catheter tip end annular recess 1310.

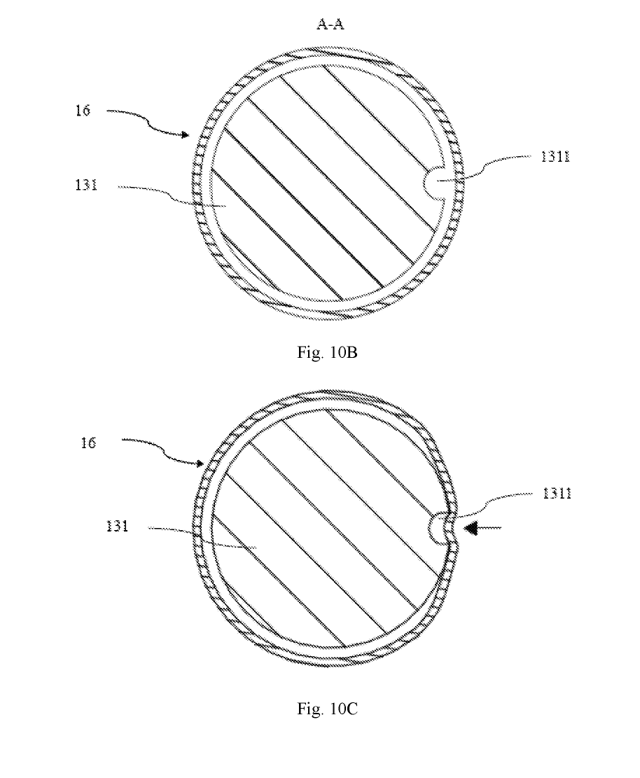

As shown in FIGS. 10A, 10B and 10C, when the thin segment inner cavity route inner opening 1311 of the urinary catheter tip end 131 of the utility model is designed in isolation, during the filling process of the thin segment 16, the area of the thin segment 16 contacted by the thin segment cavity route inner opening 1311 is too small to overcome the vertical pressure of the urethral wall at the stricture of the urethra, with the pressure direction shown by the arrow in FIG. 10C, and the thin segment inner cavity route inner opening is provided in the urinary catheter tip end annular recess 1310, so that the fluid can contact the thin segment dilatable part 1612 with a larger area during filling, which is easier to fill.

### Embodiment 8

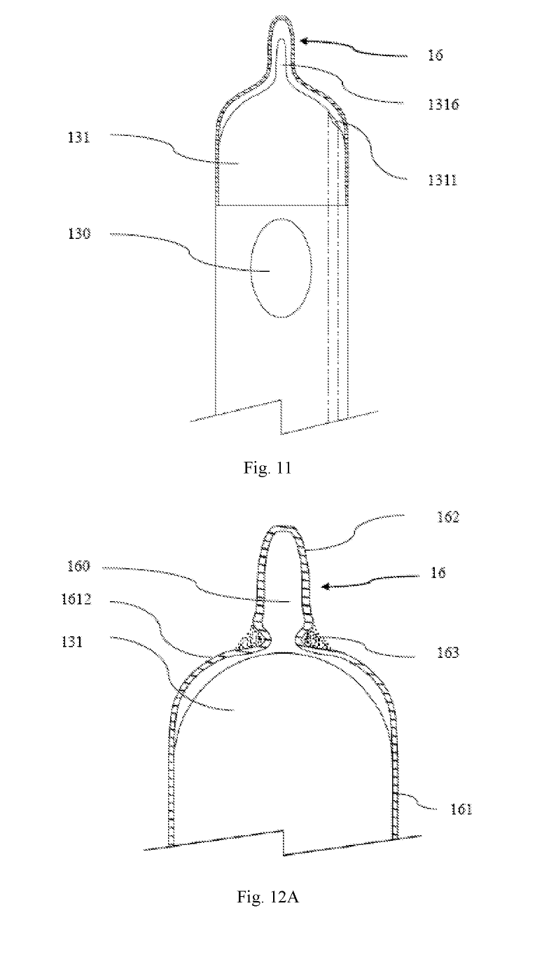

As shown in FIG. 11, in the present invention, since the thin segment 16 is of flexible design and must have the ability to enter the urethral stricture first, a part 1316 of the urinary catheter tip end 131 of this embodiment extends into the thin segment inner cavity, thereby increasing the external strength of the thin segment 16 without increasing or significantly increasing the outer diameter of the thin segment 16, which facilitates its smooth entry into the urethral stricture.

### Embodiment 9

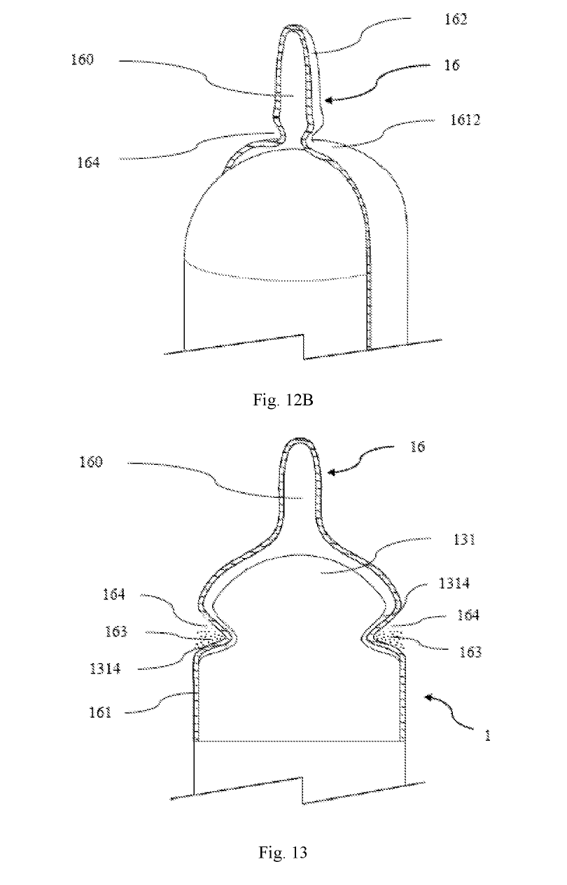

As shown in FIGS. 12A and 12B, the outer surface of the thin segment dilatable part 1612 is provided with an inward thin segment annular recess 164. When the thin segment 16 is filled and expanded, lubricating substances such as glycerin, paraffin oil and the like stored or added here and/or drugs 163 can be annularly and evenly coated on the urethral intima in the stricture, and the auxiliary expansion effect is more conducive to the smooth passage of the urinary catheter due to the accurate lubrication of the lubricating substances in the urethral intima in the stricture. However, the directional application of drugs such as anti-infection and promoting mucosal growth has a correspondingly high clinical value even if they are not used for dilatation of narrow urethra.

### Embodiment 10

As shown in FIG. 13, for the synchronous conformal recess 1314 of the urinary catheter tip end 131 and the thin segment 16 in this embodiment, in order to prevent the thin segment 16 from being softer, lubricating substances and/or drugs 163 stored or added in the thin segment annular recess 164 as described in Embodiment 9 may be lost due to the easy deformation of the thin segment 16 during the travel of the catheter 1. At the same time, the synchronous conformal recess 1314 of the urinary catheter tip end 131 and the thin segment 16 can also relatively increase the volume of the thin segment annular recess 164, in which more lubricating substances and/or drugs 163 can be stored and added.

What is claimed is:

1. A stricture-dilated urinary catheter, which is a main tube having an inner surface (11) and an outer surface (12), comprising a urinary catheter tip (13), a urinary catheter tail (14) and a urinary catheter middle section (15), in which, the tube inside is provided with a urinary catheter inner cavity (10) and the urinary catheter tip (13) is provided with a urine inlet (130) and the urinary catheter tail (14) is provided with a urine outlet (140); it is characterized in that a nipple-shaped flexible and hollow thin segment (16) which comprises a thin segment bottom (161) connected to the urinary catheter tip end (131) and a thin segment top (162) far away from the urinary catheter tip end (131); the thin segment top (162) is closed to the outside by the blind end, and the average outer diameter of the part between the thin segment top farthest point (162p) and the urinary catheter tip end farthest point (131p) is smaller than the maximum outer diameter of the urinary catheter tip end (131); the part of the thin segment (16) connected with outer surface of the urinary catheter tip end (131) is the thin segment connecting part, and the rest part is the thin segment dilatable part (1612); the thin segment (16) inside is provided with a thin segment inner cavity (160) which is specifically formed by the inner surface of the thin segment dilatable part (1612) and a portion (1315) of the outer surface of the urinary catheter tip end (131) covered by the thin segment dilatable part (1612); the thin segment inner cavity (160) is connected to the outside through at least one thin segment inner cavity route (1312), which has at least one thin segment inner cavity route inner opening (1311) and at least one thin segment inner cavity route outer opening (1313) at the urinary catheter middle section (15) or tail (14).

2. A stricture-dilated urinary catheter according to claim 1, wherein, in addition to the main drainage branch (141) connected with the urinary catheter inner cavity (10), the urinary catheter tail (14) further comprises a branch, i.e. thin segment filling collateral branch (143), connected with the thin segment inner cavity route (1312). A hollow elastic balloon (17) is connected to the end of the thin segment filling collateral branch (143), and the elastic balloon inner cavity (170) is connected with the thin segment inner cavity route outer opening (1313).

3. A stricture-dilated urinary catheter according to claim 1, wherein, a hollow elastic balloon (17) is sleeved on the urinary catheter middle section (15) near the urinary catheter tail (14) or the outer surface of the main drainage branch (141), and the elastic balloon inner cavity (170) is connected with the thin segment cavity route outer opening (1313), that is, the thin segment cavity route outer opening (1313) is provided on the urinary catheter outer surface (12) closed and covered by the elastic balloon (17).

4. A stricture-dilated urinary catheter according to claim 2, wherein, the elastic balloon (17) is provided with at least one elastic balloon external opening (171) which can be covered by fingers, and the elastic balloon inner cavity (170) is connected with the outside through this opening.

5. A stricture-dilated urinary catheter according to claim 4, wherein, a elastic balloon external opening (172) is further provided on the opposite side of the elastic balloon external opening (171).

6. A stricture-dilated urinary catheter according to claim 2, wherein, a switching part (3) which is not easy to deform under pressure and can move relative to the outer wall of the urinary catheter is provided on or near the elastic balloon (17), and a protrusion (31) which can close another external opening of the elastic balloon (173) is provided on the switching part (3); when the position of the switching part (3) is moved, the closing protrusion (31) thereon can release the closing of another external opening of the elastic balloon (173), and enable the elastic balloon inner cavity (170) to connect with the outside through a hidden route (30) on the switching part (3).

7. A stricture-dilated urinary catheter according to claim 1, wherein, at least one inward urinary catheter tip end annular recess (1310) is provided on the outer surface of the portion (1315) where the urinary catheter tip end (131) is covered by the thin segment dilatable part (1612), and a thin segment inner cavity route inner opening (1311) is provided in the urinary catheter tip end annular recess (1310).

8. A stricture-dilated urinary catheter according to claim 1, wherein, a portion (1316) of the urinary catheter tip end (131) extends into the thin segment inner cavity (160).

9. A stricture-dilated urinary catheter according to claim 1, wherein, at least one inward thin segment annular recess (164) is provided on the outer surface of the thin segment dilatable part (1612).

10. A stricture-dilated urinary catheter according to claim 1, wherein, at least one inward urinary catheter tip end annular conformal recess (1314) is provided on the portion (1315) where the urinary catheter tip end (131) is covered by the thin segment dilatable part (1612), and the thin segment dilatable part (1612) also synchronizes with the thin segment annular recess (164) at this annular conformal recess (1314).
